SEQUENCE LISTING (12 sequences)

SEQ ID NO: 1 - DNA, 65 bp, Artificial Sequence
  PCR forward primer TR05
taattaaaatatacttttgtaggcctcaaaaatccatatacgcaccggatccccgggttaattaa

SEQ ID NO: 2 - DNA, 65 bp, Artificial Sequence
  PCR reverse primer TR06
aacgaacatgttggaattgtatcattagaatacaactggaagagcgaattcgagctcgtttaaac

SEQ ID NO: 3 - DNA, 20 bp, Artificial Sequence
  PCR primer TR019
aggaatgtccgtgttcgaat

SEQ ID NO: 4 - DNA, 65 bp, Artificial Sequence
  PCR forward primer TR09
gaagacgtttatagacataaataaagaggaaacgcattccgtggtcggatccccgggttaattaa

SEQ ID NO: 5 - DNA, 65 bp, Artificial Sequence
  PCR reverse primer TR010
tattataatactcaacacgtgacttcagccttcccaattaataaagaattcgagctcgtttaaac

SEQ ID NO: 6 - DNA, 21 bp, Artificial Sequence
  PCR primer TR021
tctcggcttcggactctttta

SEQ ID NO: 7 - DNA, 65 bp, Artificial Sequence
  PCR forward primer TR053
tttatagcggaaccgctttctttatttgaattgtcttgttcaccacggatccccgggttaattaa

SEQ ID NO: 8 - DNA, 65 bp, Artificial Sequence
  PCR reverse primer TR054
cataaattcaaaaattatattatatcaggttgcatctggaaggttgaattcgagctcgtttaaac

SEQ ID NO: 9 - DNA, 21 bp, Artificial Sequence
  PCR primer TR060
ggtctcgacaccataaatcga

SEQ ID NO: 10 - DNA, 65 bp, Artificial Sequence
  PCR forward primer TR055
ccacttaataaagctagtatgataagatcaccggtagacgaaatacggatccccgggttaattaa

SEQ ID NO: 11 - DNA, 65 bp, Artificial Sequence
  PCR reverse primer TR056
aaagaattaataagtagccctttttcaaattatgtgtgcatccaggaattcgagctcgtttaaac

SEQ ID NO: 12 - DNA, 21 bp, Artificial Sequence
  PCR primer TR061
ccctcccccttttaaagtaat

(19) United States
(12) Patent Application Publication
(10) Pub. No.: US 2002/0160444 A1
(43) Pub. Date: Oct. 31, 2002

(54) Control of biofilm development

(75) Inventors: Todd B. Reynolds, Somerville, MA (US); Gerald R. Fink, Chestnut Hill, MA (US)

Correspondence Address: Law Offices of Wayne A Keown, Wayne A Keown Esq, 500 West Cummings Park, Suite 2900, Woburn, MA, 01801, US

(73) Assignee: Whitehead Institute for Biomedical Research, Cambridge, MA (US)

(21) Appl. No.: 09/928,248

(22) Filed: Aug. 10, 2001

Related U.S. Application Data
(60) Provisional application No. 60/266,390, filed on Feb. 2, 2001.
(60) Provisional application No. 60/224,734, filed on Aug. 11, 2000.

(51) Int. Cl. C12Q 1/02; C12Q 1/18; C12P 1/00

(52) U.S. Cl. 435/41; 435/29

(57) ABSTRACT

A method of producing fungal, particularly yeast, biofilms, such as methods of producing fungal biofilms on surfaces (e.g., solid and semi-solid surfaces) and methods of using the resulting biofilms to identify drugs that alter (inhibit or enhance) biofilm formation and/or flo11 function and to identify genes and proteins necessary and/or sufficient for biofilm formation.

### RELATED APPLICATIONS

This application claims the benefit of U.S. Provisional Application No. 60/266,390, entitled “Control of Biofilm Development”, by Todd B. Reynolds and Gerald R. Fink, filed on Feb. 2, 2001 and U.S. Provisional Application No. 60/224,734, entitled “Control of Biofilm Development”, by Todd B. Reynolds and Gerald R. Fink, filed on Aug. 11, 2000. The entire teachings of the above applications are incorporated herein by reference.

### GOVERNMENT SUPPORT

Work described herein was funded, in whole or in part, by National Research Service Award IF 32 GM20565 and National Institutes of Health Grant 5 RO1 GM40266. The United States Government has certain rights in the invention.

### BACKGROUND OF THE INVENTION

Microorganisms, such as fungi, often adhere to the inert surfaces of medical devices, such as prosthetic heart valves, catheters, pacemakers, silicone voice prostheses, endotracheal tubes, cerebrospinal shunts, joint replacements and dentures, and then colonize the surfaces in the form of a biofilm that consists of one or more layers of microbial cells embedded in a matrix of polymeric material secreted by the microorganism. In this phase of growth, microorganisms are typically more resistant to antibiotics than when they are growing in a planktonic (free swimming) state and, as a result, are much more difficult to treat. Fungal infections are difficult to treat and are responsible for many deaths in hospital patients. Antifungal drug therapy is ineffective against fungal biofilms because in this state, fungi are very resistant to antifungal drugs.

The molecular basis of biofilm formation and maintenance is poorly understood. A better understanding of how pathogenic organisms form biofilms and how they adhere to inert surfaces would not only make it possible to determine what types of drugs block or interfere with biofilm formation and/or block adherence to inert surfaces, but also enable design of materials, such as those useful in prosthetic devices, that do not permit attachment of pathogenic fungi.

### SUMMARY OF THE INVENTION

The present invention relates to a method of producing a fungal biofilm, comprising culturing a fungus (e.g., yeast) in an appropriate medium that contains a non-glucose repressing carbon source (e.g., a medium in which glucose concentration is limiting), under conditions appropriate for growth of the fungus, whereby a fungal biofilm is produced. The fungus can be a pathogenic fungus or a nonpathogenic fungus.

The present invention also relates to a method of producing a fungal biofilm on a surface. In this method, a fungus in an appropriate medium that contains a non-glucose repressing carbon source is applied to a surface (e.g., a solid surface; semi-solid surface), under conditions appropriate for growth of the fungus, thereby producing a surface having applied thereto a fungus in the appropriate medium. The surface thereby produced is maintained under conditions under which fungal cells adhere to the surface and form a biofilm, thereby forming a fungal biofilm on the surface.

Fungal biofilms produced by the methods described herein are also the subject of the present invention.

Also encompassed by the present invention is a method of identifying a drug that alters (inhibits, reduces, enhances) formation of a fungal biofilm. In this embodiment, a fungus in an appropriate medium that contains a non-glucose repressing carbon source is applied to a surface, in the presence of a drug to be assessed for its ability to alter formation of a fungal biofilm and under conditions appropriate for growth of the fungus, thereby producing a surface having applied thereto a fungus in the presence of the drug and in the appropriate medium. The surface thereby produced is maintained under conditions under which fungal cells adhere to the surface and form a biofilm. The extent to which a fungal biofilm is produced is determined and compared to the extent to which a fungal biofilm is formed under the same conditions but in the absence of the drug, wherein if the extent to which the fungal biofilm is produced is different in the presence of the drug than in the absence of the drug, then the drug is a drug that alters formation of the fungal biofilm. In one embodiment, the extent to which the fungal biofilm is produced in the presence of the drug is less than the extent to which the fungal biofilm is produced in the absence of the drug and the drug is, therefore, a drug that reduces formation of the fungal biofilm. In another embodiment the extent to which the fungal biofilm is produced in the presence of the drug is greater than the extent to which the fungal biofilm is produced in the absence of the drug and the drug is, therefore, a drug that enhances formation of the fungal biofilm.

The work described herein allows for identification of drugs that block or interfere with biofilm formation and/or block adherence to inert surfaces. In addition, materials which block or retard attachment of biofilms, particularly pathogenic fungi, can be developed.

### BRIEF DESCRIPTION OF THE DRAWINGS

FIGS. 1A-1D show that Saccharomyces formed biofilms on the surface of polystyrene. FIG. 1A shows adherence to plastic at a low glucose concentration. The cells were incubated for 0, 1, 3, or 6 hours at 30° C. The time (hours) after inoculation is above the wells and glucose concentrations (%) are below. FIG. 1B shows that Flo11p was required for adherence. Yeast strains were resuspended in SC+0.1% glucose prior to adding them to the plate. All of the wild-type and mutant strains used were isogenic. The numbers at the top are min after additions to the plate. FIG. 1C shows quantitation of the results shown in FIG. 1B. Each data point is the average of 3 samples (▪MATαFLO11, ●MATaFLO11, ▴MATa/αFLO11/FLO11, □MATαflo11Δ, ◯MATaFLO11Δ, ΔMATa/αflo11 Δ/flo11 Δ.) FIG. 1D is a photograph of the cells in the wells shown in FIG. 1B. The cells were photographed at 100× magnification with a Zeiss Telaval 31 inverted microscope. Incubation in the well was for 0 min (left) and 180 min (right). The scale bar is 50 μm.

FIGS. 2A-2L show mat formation in Saccharomyces. FIGS. 2A-2G show formation of a single mat by a MATα strain over time on a 3% agar plate. The same plate was photographed after (FIG. 2A) 2, (FIG. 2B) 4, (FIG. 2C) 5, (FIG. 2D) 6, (FIG. 2E) 7, (FIG. 2F) 9 and (FIG. 2G) 13 days at 25° C. FIG. 2H shows the same MATα strain on a 2% agar YPD plate after 13 days at 25° C. FIGS. 2I-2K show that mating type affected the morphology of mats. Compare the MATa strain (FIG. 2I) with the MATα strain (FIG. 2G), both grown for 13 days on YPD-0.3% agar. FIGS. 2J and 2K show that a MATa/α diploid is delayed in spoke appearance (compare FIGS. 2E and 2J, both at 7 days of growth). By 13 days the deploid resembles the haploids (compare FIGS. 2G, 2I, and 2K). FIG. 2L shows that FLO11 function is required for mat formation. A flo11Δ strain after 13 days of growth on a 0.3% YPD plate. The scale bar is 1 cm.

FIGS. 3A-3D show the structure of the yeast mats. FIG. 3A shows the parallel cables that formed the white spokes seen in FIG. 2G (MATα). The spokes seemed to emanate from the spaghetti-like network of cables originating in the hub. The lighter color of the spoke contrasted with the smoother edge of the mat. FIG. 3B shows the flo11Δ mat (FIG. 2L) was smooth with no substructure. FIG. 3C shows the MATa strain (FIG. 2I) had a spaghetti-like network of cables that extended to the edge of the mat and formed narrower and more frequent spokes. The photographs in FIGS. 3A-3C were made at 5× magnification through a Technival 2 dissecting microscope. The scale bar is 1 mm. FIG. 3D is a scanning electron micrograph of the yeast form cells that comprise the MATα mat was made at 5500× magnification. The scale bare is 2 μm.

FIGS. 4A to 4D show that increased ploidy reduced mat formation. A series of isogenic strains from haploid to tetraploid (19) on YPD-0.3% agar plates after 13 days at 25° C. Previous work (Galitski et al., Science, 285:251 (1999)) had shown that the level of FLO11 as well as the FLO11-dependent agar invasion phenotype of Σ1278 b decreased as the ploidy increased. (FIG. 4A) MATα, (FIG. 4B) MATαα, (FIG. 4C) MATααα, (FIG. 4D) MATααα. The scale bar is 1 cm.

### DETAILED DESCRIPTION OF THE INVENTION

As described herein, Applicants have developed a fungal biofilm model system, have identified genes involved in adhesion of yeast to surfaces and have shown that biofilm formation is dependent on the presence of certain flocculins, such as FLo11p, which is a member of the same cell surface protein superfamily as the glabrata and albicans adhesins. Adhesion of yeast to inert surfaces has been shown to result from the expression of a set of cell surface glycoproteins that comprise a generic (common) domain structure. Each cell surface glycoprotein comprises an N-terminal signal sequence; and immunoglobulin-like domain; a serine-threonine rich O-glycosylated central portion and a GPI-linked carboxy-terminus. Specificity for adhesion to a target surface appears to reside in the immunoglobulin-like domain.

Work described herein has shown that certain strains of S. cerevisiae attach to inert surfaces and that S. cerevisiae exhibits formation of previously undetected biofilm-like behavior on the surface of agar plates. The behavior of S. cerevisiae is similar to that of certain pathogenic bacteria, such as Mycobacterium smegmatis. The observation that biofilm formation is dependent on the presence of certain flocculins, such as Flo11p, is important because most molecular insights into relevant pathogenic phenotypes of C. albicans have been discovered using S. cerevisiae as a surrogate (Sundstrom, P., Curr. Opin. Microbiol, 2:353-357 (1999)). The work described herein has, thus, provided a model useful for the identification of the molecules responsible for adherence of other fungi, particularly pathogenic fungi, to inert surfaces. Many observations that have been made in S. cerevisiae are easily translated into an understanding of C. albicans (Madhani and Fink, Cell 91: 673-684 (1997)) and, therefore, it is reasonable to expect that it will also be a good model for understanding adhesion to inert surfaces and biofilm formation by pathogenic fungi. In addition, adhesion proteins are a conserved gene family that is found in all fungi and, therefore, identification of the genes that cause adhesion in S. cerevisiae will permit identification of orthologous family members in pathogenic yeast, such as species of Candida (e.g., C. albicans).

Applicants have identified conditions under which S. cerevisiae adheres to an inert surface and shown that when grown under these conditions, the baker's yeast forms a tightly adhering biofilm on the surface. Under the conditions described herein, the baker's yeast forms such a biofilm on polystyrene (plastic).

Accordingly, the present invention relates to methods of producing biofilms, such as fungal biofilms and particularly yeast biofilms; methods in which the biofilms are used as a model system, such as assay methods for identifying genes and proteins necessary and/or sufficient for adhesion and biofilm formation and methods of identifying drugs that alter (inhibit, reduce, disrupt or enhance) biofilm formation, such as fungal, particularly yeast, biofilm formation; fungal biofilm model systems, such as yeast biofilm model systems; and drugs and genes identified by methods of the present invention.

As described herein, Applicants have shown that adhesion of yeast to inert surfaces strongly correlates with the expression of a set of cell surface glycoproteins that comprise a generic (common) domain structure. That is, each glycoprotein comprises an N-terminal signal sequence; an immunoglobulin-like domain; a serine-threonine rich O-glycosylated central portion and a GPI-linked carboxy-terminus. Specificity for adhesion to a target surface appears to reside in the immunoglobulin-like domain. Further, Applicants have shown that a fungus can initiate biofilm formation and form a structure referred to herein as a “mat”. When grown in medium that comprises a non-glucose repressing carbon source, such as a low-glucose medium, the fungal cells adhere avidly to plastic surfaces. On semi-solid medium, the fungal cells form mats, which are multicellular structures of considerable complexity. In a particular embodiment, yeast initiate biofilm formation and form a mat. When grown in a medium that contains a non-glucose repressing carbon source, such as a low-glucose medium, the yeast cells adhere avidly to a number of plastic surfaces and on semi-solid medium, form mats, which are multicellular structures comprised of yeast-form cells.

In one embodiment of the present invention, a fungal (e.g., a yeast) biofilm is produced by culturing a fungus in medium that contains a non-glucose repressing carbon source, under conditions appropriate for growth of the fungus, whereby a fungal (e.g., yeast) biofilm is produced. In a specific embodiment of producing a fungal (e.g., yeast) biofilm, the fungus (e.g., yeast) is cultured in an appropriate medium in which glucose concentration is limiting, under conditions appropriate for growth of the fungus (e.g., yeast), whereby a fungal (e.g., yeast) biofilm is produced. In specific embodiments, the fungus is yeast. In a further embodiment, the yeast is in mid log phase and the appropriate medium is synthetic complete medium. In another embodiment, the yeast is mid log phase, the appropriate medium is synthetic complete medium and the limiting glucose concentration is 0.1%. In all embodiments, the fungus can be any of a variety of fungi and can be pathogenic or nonpathogenic. The yeast can be nonpathogenic (e.g., Saccharomyces (S.) cerevisiae) or pathogenic (e.g., Candida (C.) albicans). The fungus can be filamentous fungus, such as, but not limited to, members of the following: Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species.

Another embodiment of the present invention is a method of producing a fungal biofilm on a surface, comprising applying a fungus in an appropriate medium that comprises a non-glucose repressing carbon source, such as a low-glucose medium, to a surface, thereby producing a surface having applied thereto the fungus in an appropriate medium and maintaining the surface thereby produced under conditions under which fungal cells adhere to the surface and form a biofilm, thereby forming a fungal biofilm on the surface. The fungus can be any of a variety of fungi and can be pathogenic or nonpathogenic. The surface to which the fungus in medium is applied can be any of a variety of types of materials, including solid and semi-solid surfaces and inert/nonporous and porous surfaces, as described further herein. In specific embodiments, the fungus is yeast. In a further embodiment, the yeast is in mid log phase and the appropriate medium is synthetic complete medium. In another embodiment, the yeast is in mid log phase, the appropriate medium is synthetic complete medium and the limiting glucose concentration is 0.1%. In all embodiments, the fungus can be any of a variety of fungi and can be pathogenic or nonpathogenic. The yeast can be nonpathogenic (e.g., Saccharomyces (S.) cerevisiae) or pathogenic (e.g., Candida (C.) albicans). The fungus can be filamentous fungus, such as, but not limited to members of the following: Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species.

In a further embodiment of producing a fungal biofilm, such as a yeast biofilm, an appropriate medium that comprises a non-glucose repressing carbon source, such as a low-glucose medium, is applied to a surface and the fungus from which the fungal biofilm is to be produced is added to the medium, thereby producing a surface bearing the medium and the fungus. The resulting surface is maintained under appropriate conditions for growth of the fungus and production of a biofilm, whereby a fungal biofilm is produced. In this embodiment as well, the fungus can be any of a variety of fungi and can be pathogenic or nonpathogenic. The surface to which the fungus in medium is applied can by any of a variety of types of materials, including solid and semi-solid surfaces, biotic or abiotic surfaces, nonporous and porous surfaces, as described further herein. In specific embodiments, the fungus is yeast. In a further specific embodiment, the yeast is in mid log phase and the appropriate medium is synthetic complete medium. In another embodiment, the yeast is in mid log phase, the appropriate medium is synthetic complete medium and the limiting glucose concentration is 0.1%. In all embodiments, the fungus can be any of a variety of fungi and can be pathogenic or nonpathogenic. The yeast can be nonpathogenic (e.g., Saccharomyces (S.) cerevisiae) or pathogenic (e.g., Candida (C.) albicans). The fungus can be filamentous fungus, such as, but not limited to, a member of the following: Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species.

One embodiment of the present invention is a method of producing a fungal biofilm on a surface, comprising: (a) applying a fungus in an appropriate medium that contains a non-glucose repressing carbon source, under conditions appropriate for growth of the fungus, thereby producing a surface having applied thereto a fungus in the appropriate medium; and (b) maintaining the surface thereby produced under conditions under which fungal cells adhere to the surface and form a biofilm, thereby forming a fungal biofilm on the surface. In a specific embodiment, the fungus is yeast, such as filamentous.

According to a further embodiment of the method of the present invention, a fungal (e.g., yeast) biofilm is produced on a surface, such as solid surface, by:

(a) applying a fungus (e.g., yeast, such as yeast in mid log phase) in an appropriate media (e.g., synthetic complete (SC) media) in which glucose concentration is limiting to a solid surface, thereby producing a surface having applied thereto a fungus (e.g., yeast) in appropriate media; and

(b) maintaining the surface thereby produced under conditions under which fungal (e.g., yeast) cells adhere to the solid surface and form a biofilm, thereby forming a fungal (e.g., yeast) biofilm on the solid surface.

In a further embodiment, steps (a) and (b) are carried out as above, followed by:

(c) removing loose cells (cells, such as yeast cells, not adhered to the solid surface) from the solid surface, thereby producing a solid surface having cells (e.g., yeast cells) adhered thereto and loose (nonadhered) cells removed therefrom; and

(d) submerging the solid surface thereby produced in a low concentration glucose solution under conditions appropriate for fungal (e.g., yeast cell) growth, thereby producing a fungal (e.g., yeast) biofilm on the solid surface.

In one embodiment, the limiting glucose concentration is from about 0.1% to about 2.0% generally from about 0.1% to about 0.6% (from about 5 mM to about 35 mM glucose) and, in a specific embodiment, is about 0.1% or about 0.3% glucose (from about 5 m M to about 15 mM glucose). Loose cells can be removed by any appropriate method, such as by washing with water (e.g., with sterile double deionized water). The solid surface having yeast cells adhered thereto and loose cells removed therefrom is submerged in 0.1% glucose for sufficient time (e.g., as long as is sufficient to permit the desired extent of or adequate cell growth, such as 6 hours or overnight).

The solid surface on which the fungal (e.g., yeast) biofilm is produced can be any of a wide variety of materials, particularly inert materials, including, but not limited to, plastic, polystyrene, polyvinylchloride and polypropylene. They can be in any shape, such as a flat surface, a well, a tube or a sphere. The fungus which forms the biofilm can be nonpathogenic, such as Saccharomyces (e.g., S. cerevisiae), or pathogenic, such as any Candida species (e.g., C. albicans).

A particular embodiment of producing a fungal, such as a yeast, biofilm on an inert surface, wherein the inert surface is plastic/polystyrene, comprises the following steps:

(a) growing the yeast to mid-log phase in SC 2% glucose (1-2 OD/mL) or in other appropriate media and a similar glucose concentration;

(b) washing the product of (a) one or more times with sterile double deionized water, thereby producing washed yeast cells;

(c) resuspending the washed yeast cells to about 1 OD/mL in SC 0.1% glucose;

(d) adding 100λ of cells/well to a welled plate (e.g., 24, 48 96 wells), thereby producing wells containing yeast cells in SC 0.1% glucose;

(e) maintaining wells containing yeast cells produced in (d) for sufficient time (e.g., 1 hour to several days, e.g., 1, 3, 6, 8 hours, overnight or several days such as 1 to 24 days) and at an appropriate temperature (e.g., about 25° C., about 30° C. or a temperature there between) for yeast cell growth to occur, thereby producing a yeast biofilm on the surface; and, optionally,

(f) assaying yeast cell growth by adding a sufficient amount of an appropriate dye, such as 100λ crystal violet, to each well and maintaining the resulting combination for sufficient time (e.g., 15 minutes or longer) for dye uptake into or binding to cells or cell components to occur;

(g) washing the product of (f), such as three times with double deionized water; and

(h) assessing the extent to which the dye (e.g., crystal violet) is present in each well, thereby providing an indication of the extent to which yeast cells are present in each well, wherein the larger the amount or concentration of dye present in a well, the greater the extent to which cells are present in the well.

In further embodiment of the present invention, in which a biofilm is produced on polystyrene or other plastic, the surface is preferably sterilized (e.g., with ethanol) and approximately 100λ of yeast cells suspension, grown, washed and resuspended as described above, are spotted onto the surface. The resulting surface, which bears the yeast cell suspension, is maintained at an appropriate temperature, such as about 30° C., for sufficient time (e.g., 1 hour or longer) for cells to adhere and grow and then washed (e.g., 3 times with sterile double deionized water). The resulting product is placed in SC 0.1% glucose (e.g., 5 mL) and allowed to grow (maintained under conditions appropriate for growth) for as long as needed to attain the desired growth (e.g., overnight). As described herein, Applicants have shown that adhesion of yeast to inert surfaces results from the expression of a set of cell surface glycoproteins that comprise a generic (common) domain structure. That is, each glycoprotein comprises an N-terminal signal sequence; an immunoglobulin-like domain; a serine threonine rich O-glycosylated central portion and a GPI-linked carboxy-terminus. Specificity for adhesion to a target surface appears to reside in the immunoglobulin-like domain.

As a result of the work described herein, methods of identifying drugs (compounds or molecules) that alter adhesion and biofilm formation by microorganisms, particularly fungi such as yeast, are available, as are methods of identifying genes and proteins necessary and/or sufficient for biofilm formation, such as fungal, particularly yeast, biofilm formation. They are particularly useful to identify drugs that inhibit (reduce, prevent or reverse) biofilm formation, such as those that develop on inert surfaces of medical devices, and to identify genes and/or proteins that are targets, particularly specific targets, for inhibitors.

Thus, a further embodiment of the present invention is a method of identifying a drug that alters formation of a fungal biofilm. In this method, a fungus in an appropriate medium that contains a non-glucose repressing carbon source is applied to a surface, in the presence of a drug to be assessed for its ability to alter formation of a fungal biofilm and under conditions appropriate for growth of the fungus, thereby producing a surface having applied thereto a fungus in the presence of the drug and in the appropriate medium. The surface thereby produced is maintained under conditions under which fungal cells adhere to the surface and form a biofilm; and the extent to which a fungal biofilm is produced is determined, and compared to the extent to which a fungal biofilm is formed under the same conditions but in the absence of the drug, wherein if the extent to which the fungal biofilm is produced is different in the presence of the drug than in the absence of the drug, then the drug is a drug that alters formation of the fungal biofilm. If the extent to which the fungal biofilm is produced in the presence of the drug is less than the extent to which the fungal biofilm is produced in the absence of the drug, then the drug reduces (e.g., inhibits partially or completely) formation of the fungal biofilm. If the extent to which the fungal biofilm is produced in the presence of the drug is greater than the extent to which the fungal biofilm is produced in the absence of the drug, then the drug enhances formation of the fungal biofilm. In one embodiment, the fungus is a pathogenic fungus. In another embodiment, the fungus is a nonpathogenic fungus. In yet another embodiment, the fungus is a filamentous fungus (e.g., Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species).

Exemplification

Methods and Materials

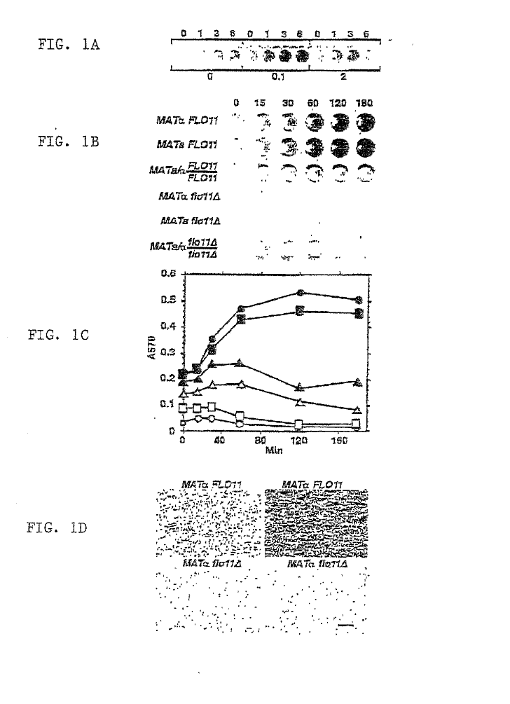

Synthetic complete (SC) media and plates and YPD plates were made as in Guthrie, C. and Fink, G. R., Methods in Enxymol., 194:12 (1991) with the exception of the altered agar or glucose concentrations. Yeast was grown in SC with 2% (w/v) glucose and harvested at an optical density at 600 nm (OD₆₀₀ₙₘ) of 0.5-1.5. Cells were then washed once in sterile H₂O, resuspended to 1.0 OD₆₀₀ₙₘ in SC with 0, 0.1%, or 2% glucose, and pipetted (100 μl) into wells of a 96 well polystyrene plate (Falcon Microtest flat bottom plate, 35-1172; Becton-Dickinson Labware). Cells that adhered to polystyrene were visualized by staining with crystal violet (For visualization of biofilms in wells of polystyrene plates a protocol was adapted from (G. A. O'Toole et al, Methods Enzymol. 310, 91 (1999)), an equal volume of a solution of 1% (w/v) crystal violet was added to the cells and left for at least 15 min. The wells were washed repeatedly with H₂O and then photographed. Cells were also observed to adhere to polystyrene when not dyed with crystal violet.) FIG. 1A shows adherence to plastic at a low glucose concentration. The cells were incubated for 0, 1, 3, or 6 hours at 30° C. The time (hours) after inoculation is above the wells and glucose concentrations (%) are below. FIG. 1B shows that Flo11p was required for adherence. Yeast strains were resuspended in SC+0.1% glucose prior to adding them to the plate. All of the wild-type and mutant strains used were isogenic. (An isogenic MATαflo11Δ strain (TBR5) was generated in TBR1 (Σ1278 b strain 10560-23C; MATα, ura3-52, his3::hisG, leu2::hisG) by protocol of (M. S. Longtine, et al., Yeast 14, 953 (1998)). A polymerase chain reaction (PCR) fragment was used to replace the entire FLO11 open reading frame with the kanamycin resistance gene. Other disruptants were generated in a similar manner (primers used for each disruptant can be found in Science on-line). The isogenic MATa strain (TBR2) was generated by transforming TBR1 with the pGAL::HO plasmid (B1377) and switching the mating type (Galitski et al., Science, 285:251 (1999)). The diploid MATa/α strain (TBR3) was generated by crossing TBR1 to TBR2. The MATaflo11Δ strain (TBR12) was generated by crossing TBR1 and TBR5 and sporulating the diploid and the MATa/αflo11Δ/flo11Δ strain (TBR13) was generated by crossing TBR5 and TBR12.

Isogenic yeast strains were inoculated onto YPD agar plates (0.3% or 2%) with a toothpick 1 to 2 days after the plates were poured. The plates were then wrapped with parafilm and incubated at 25° C.

The adherence of cells was quantitated by solubilizing the crystal violet with 100 μl of 10% SDS. After 30 min 100 μl of H₂O were added, the solution was mixed by pipetting, and 50 μl were transferred to a fresh polystyrene 96 well plate (Flat-bottom Nunc-MicroWell plate, 269620, Nalge Nune International). The absorbance at 570 nm (A570) was then read using a Dynatech MR600 microplate reader. In some experiments, after the cells were added the plates were spun at 3000 rpm for 5 min prior to the assay. This centrifugation controlled for the possibility that the differences observed between strains were due to differences in the rate at which the cells settled to the bottom of the plate. The general trends shown in FIGS. 1B and 1C were observed regardless of whether the plates were spun before the assay.

Primers used in constructing isogenic strains: flo11Δ: The primers used to generate the PCR disruption fragment for FLO11 from the pFA6a-kanMX6 plasmid were: Forward: TRO5,5′-TAATTAAAATATACTTTTGTAGGCCTCAAAAATCCATATACGCACCGGATCC CCGGGTTAATTAA-3′ (SEQ ID NO.: 1); Reverse: TRO6, 5′-AACGAACATGTTGGAATTGTATCATTAGAATACAACTGGAAGAGCGAATTC GAGCTCGTTTAAAC-3′ (SEQ ID NO.: 2). The primers used to check the success of the disruption by PCR were: TRO19, 5′-AGGAATGTCCGTGTTCGAAT-3′ (SEQ ID NO.:3) and TRO6 above. A successful disruption gave a product of 1845 base pairs (bp). Unsuccessful disruptions gave no product. All of other disruptions used were generated in the same manner unless otherwise stated. Primers used for these disruptions are as follows:

| | |
|---|---|
| flo8Δ: | |
| Forward: | TRO9, 5′-GAAGACGTTTATAGACATAAATAAAGAGGAAACGCATTCCGTGGTCGGATC |
| | CCCGGGTTAATTAA-3′ (SEQ ID NO.: 4) |
| Reverse: | TRO10, 5′-TATTATAATACTCAACACGTGACTTCAGCCTTCCCAATTAATAAAGAATTCG |
| | AGCTCGTTTAAAC-3′ (SEQ ID NO.: 5) checked with |
| | TRO21, 5′-TCTCGGCTTCGGACTCTTTTA-3′ (SEQ ID NO.: 6) and TRO10. |
| ste12Δ: | |
| Forward: | TRO53, 5′-TTTATAGCGGAACCGCTTTCTTTATTTGAATTGTCTTGTTCACCACGGATCCC |
| | CGGGTTAATTAA-3′ (SEQ ID NO.: 7) |
| Reverse: | TRO54, 5′-CATAAATTCAAAAATTATATTATATCAGGTTGCATCTGGAAGGTTGAATTCG |
| | AGCTCGTTTAAAC-3′ (SEQ ID NO.: 8) Checked with |
| | TRO60, 5′-GGTCTCGACACCATAAATCGA-3′ (SEQ ID NO.: 9) and TRO54. |
| ste11Δ: | |
| Forward: | TRO55, 5′-CCACTTAATAAAGCTAGTATGATAAGATCACCGGTAGACGAAATACGGATC |
| | CCCGGGTTAATTAA-3′ (SEQ ID NO.: 10) |
| Reverse: | TRO56, 5′-AAAGAATTAATAAGTAGCCCTTTTTCAAATTATGTGTGCATCCAGGAATTCG |
| | AGCTCGTTTAAAC-3′ (SEQ ID NO.: 11) Checked with |
| | TRO61, 5′-CCCTCCCCCTTTTAAAGTAAT-3′ (SEQ ID NO.: 12) and TRO56. |

Results

Little is known about fungal biofilms because many of the organisms that form these structures are not amenable to genetic approaches (G. A. O'Toole, H. B. Kaplan, R. Kolter, Annu., Rev. Microbiol. 54, 49 (2000); G. S. Baillie and L. J. Douglas, Methods Enzymol. 310, 644 (1999)). The well-characterized bakers' yeast S. cerevisiae was investigated in order to develop a model system for fungal biofilms. Specifically, the ability of S. cerevisiae to form biofilms was assessed. Bacteria are said to form biofilms if they adhere to plastic (G. A. O'Toole, H. B. Kaplan, R. Kolter, Annu., Rev. Microbiol. 54, 49 (2000)). Applicants found that by this definition, S. cerevisiae can initiate biofilm formation on polystyrene plates (FIGS. 1A-1D). This film was not removed after repeated washes. The adherence of yeast to plastic was enhanced as the glucose concentration was lowered, but it was reduced in the complete absence of glucose, indicating that there is a requirement for active metabolism (FIG. 1A). Diploid cells did not adhere as well as haploids in this assay (FIGS. 1B and 1C). Examination of the attached cells by microscopy revealed that they were round yeast form cells (FIG. 1D). The cells also adhered to polypropylene and more poorly to polyvinylchloride (PVC). (PVC 96 well plates (Falcon Microtest III flexible assay plates, 35-3911; Becton Dickinson) were used to test adhesion by essentially the same assay used for the polystyrene plates. Polypropylene microfuge tubes (Eppendorf flex-tube, 1.5 ml) were used to test adhesion to this type of plastic. In this case 400 μl of cells were used because the results were easier to judge macroscopically.)

Bacterial biofilm formation requires cell surface adhesins (G. A. O'Toole, H. B. Kaplan, R. Kolter, Annu., Rev. Microbiol. 54, 49 (2000)). Therefore, FLO11, a yeast gene encoding a cell surface glycoprotein that is required for adhesion to agar (W. S. Lo and A. M. Dranginis, Mol. Biol. Cell 9, 161 (1998); B. Guo, C. A. Styles, Q. Feng, G. R. Fink, Proc. Nat. Acad. Sci. U.S.A. 97, 12158 (2000)) and FLO8, a yeast gene that encodes a regulatory protein required for FLO11 expression (H. Liu, C. A. Styles, G. R. Fink, Genetics 144, 967 (1996)) were disrupted. Isogenic strains lacking either FLO11 (flo11Δ, FIGS. 1B-1D) or FLO8 (flo8Δ) adhere very poorly to polystyrene even in low glucose. Adherence to plastic is only the initial stage of biofilm formation. In some organisms such as P. aeruginosa the biofilm matures to form microcolonies of bacteria that are surrounded by an extracellular matrix (G. A. O'Toole, H. B. Kaplan, R. Kolter, Annu., Rev. Microbiol. 54, 49 (2000)). Whether the film of yeast cells that forms on microtiter plates is a prelude to further developmental events can be assessed, using known techniques. However, the behavior of Saccharomyces in another model of biofilm formation (G. S. Baillie and L. J. Douglas, Methods Enzymol. 310, 644 (1999)), indicates that this fungus likely undergoes some subsequent steps in the process. In this assay, FLO11 strains formed a dense film on plastic rectangles submerged in liquid medium. (FLO11 or flo11Δ cells were grown in SC+2% glucose, harvested at an OD₆₀₀ₙₘ of 0.5-1.5, washed in water, and resuspended in SC+0.1% glucose to an OD₆₀₀ₙₘ of 1.0. This cell suspension (30 μl) was then placed on a small rectangle (approximately 5×5 mm) of ethanol sterilized polystyrene (cut from petri dishes) and incubated in a petri dish at 30° C. for 1.5 hours. The rectangle was then placed into a sterile well (Costar 3526.24 well Cell Culture Cluster, Corning Incorporated) containing 2 ml of SC+0.1% glucose and grown for 18-24 hours at 30° C. The plastic rectangles were removed from the media, washed gently with water, and viewed under the microscope. The FLO11 cell mass adhered to the rectangle whereas the flo11Δ cells did not. The FLO11 film, comprised of both round and elongated cells, adhered to the disks after gentle washing whereas flo11Δ cells washed off.

The role of Flo11p in the adherence of Saccharomyces to plastic may be similar to that of the glycopeptidolipids (GPLs) expressed on the cell surface of Mycobacterium smegmatis, a non-flagellated bacterium. M. smegmatis mutants defective in GPL synthesis are defective in both biofilm formation and in a distinct colonial behavior called “sliding motility”, indicating an intimate connection between the two phenotypes (J. Recht, A. Martinez, S. Torello, R. Kolter, J. Bacteriol., 182: 4348 (2000); A. Martinez, S. Torello, R. Kolter, J. Bacteriol., 181:7331 (1999)). Sliding motility is defined as a form of surface motility “. . . produced by the expansive forces of the growing bacterial population in combination with cell surface properties that favor reduced friction between the cells and the substrate” (J. Recht, A. Martinez, S. Torello, R. Kolter, J. Bacteriol., 182: 4348 (2000)).

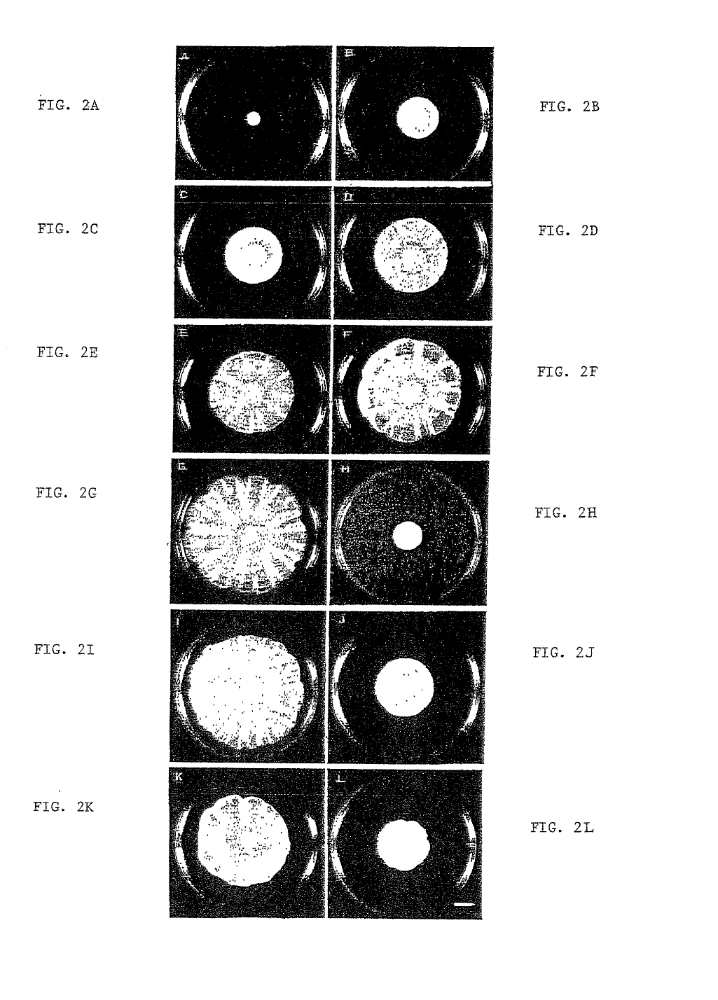

To determine whether Saccharomyces displays a FLO11-dependent phenotype similar to sliding motility, strains were inoculated onto YPD plates made with 0.3% agar instead of the 2%. On this low agar concentration, S. cerevisiae exhibited an elaborate pattern of FLO11-dependent multicellular growth resulting in a confluent mat (FIGS. 2A-2L). The low concentration of agar required for formation of this structure is similar to that which evinces the sliding motility of M. smegmatis (J. Recht, A. Martinez, S. Torello, R. Kolter, J. Bacteriol., 182: 4348 (2000); A. Martinez, S. Torello, R. Kolter, J. Bacteriol., 181: 7331 (1999)).

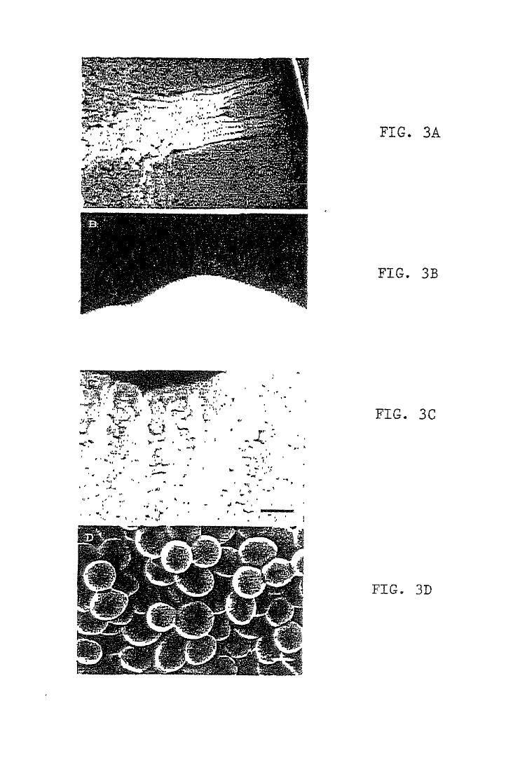

When inoculated in the center of low agar plates, S. cerevisiae produced a flat mat that covered a larger surface than that of the same strain inoculated on 2% agar (FIGS. 2G and 2H). This structure grew in a radial form both on circular and square petri dishes and ultimately covered most of the agar, achieving a diameter of 7.8+/−0.57 cm after 13 days. (The results were obtained by growing 11 independent mats and measuring the diameter and number of spokes for each mat daily.) The mature structure had a central hub made of spaghetti-like cables (FIG. 3A), and radial spokes emanating from the hub. Spokes formed reproducibly within a defined range with a mean of 14.4 spokes +/−4.5. The spokes and hub were much more distinct at 25° C. than they were at 30° C. The number of cells produced by mat formation on 0.3% agar was seven times greater (day 12) than that in a colony produced on 2% agar by the same strain. (A FLO11 MATα strain grown for 12 days at 25° C. produced an average of 8.1×10⁹ cells on YPD-0.3% agar and an average of 1.1×10⁹ cells when grown on YPD-2% agar under the same conditions. A MATαflo11Δ strain grown for 12 days at 25° C. produced an average of 5.3×10⁹ cells on YPD-0.3% agar and an average of 1.6×10⁹ cells when grown on YPD-2% agar. Cell number was determined made by transferring the cells from plates to 15 ml conical tubes (Falcon 35-2096) or microfuge tubes. The cell mass was suspended in water, the suspension was diluted, and the OD₆₀₀ₙₘ was measured. The number represents the average of three plates for each strain at each agar concentration.) The formation of mats and spokes, like adherence to plastic, is affected by glucose concentration; reduction in the glucose concentration resulted in a more rapid formation of spokes and hubs. (The mats formed on plates containing 0.25% glucose (low glucose) display mature spokes and hubs by day 4 and form a mat that is smaller in diameter than the mats formed on 2% glucose plates.)

The ability of S. cerevisiae to form the floral mat was FLO11-dependent. Growth of a flo11Δ strain on a 0.3% agar plate produced a mass of cells with a smaller diameter and without the characteristic morphology of FLO11 mat (FIGS. 2L, 3B). The FLO11 mat after 12 days on 0.3% agar had about 1.5× the number of cells as a flo11Δ strain, whereas on 2% agar a flo11Δ strain produced a colony with ˜1.4× as many cells as an isogenic FLO11 strain. FLO8, a regulator of FLO11 expression, was also required for mat formation.

The FLO11 gene is also required for filamentous growth, a morphological switch from the yeast form to multicellular pseudohyphae (invasive chains of elongated cells), that is induced by conditions of nitrogen starvation. Filamentous growth requires components of a signaling cascade of the mitogen-activated protein kinase (MAP kinase) family for maximal transcription of FLO11 (S. Rupp, E. Summers, H. J. Lo, H. Madhani, G. R. Fink, EMBO J 18, 1257 (1999); R. L. Roberts and G. R. Fink, Genes Dev. 15, 2974 (1994); H. Liu, C. A. Styles, G. R. Fink, Science 10,1741 (1993)). Mutations in genes encoding components of this MAP kinase pathway (e.g. the MAP kinase kinase kinase ste11 and the transcription factor ste12) that reduce filamentation also formed mats more slowly than wild type. Although FLO11 expression is required for both filamentous growth and mat formation, the cells in both FLO11 and flo11Δ strains were yeast form and not pseudohyphal (FIG. 3D) as determined by light microscopy and scanning electron microscopy.

The mats formed by isogenic MATα, MATα and MATα/α diploid strains had distinguishable morphologies. The mats of the MATa strain were typically smaller in diameter and formed more spokes than the MATα strain. In addition, mats formed by the MATα strain were rougher in texture with more spaghetti-like folds, rougher edges and fewer lobes than mats formed by the MATα strain (compare FIGS. 2I and 2G and FIGS. 3A and 3C).

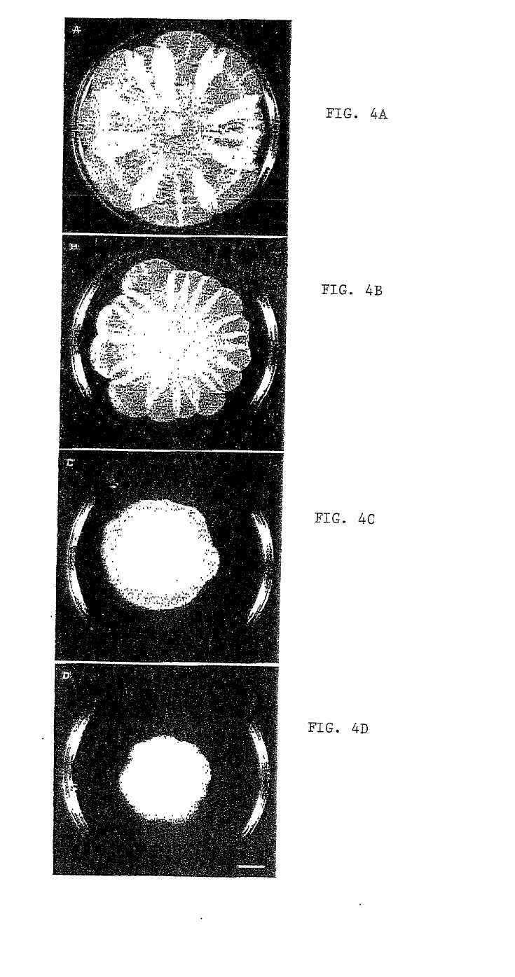

At early time points, the mats formed by the MATα/α diploid were dramatically different in morphology from that of either haploid (compare FIGS. 2J and 2E) although the differences lessened with time (compare FIGS. 2G, 2I, and 2K). This difference between haploids and diploids is likely to reflect the reduced expression of FLO11 in MATα/α diploids versus MATα or MATα haploids. Previous work has shown that FLO11 transcription decreases with increasing ploidy (T. Galitski, A. J. Saldanha, C. A. Styles, E. S. Lander, G. R. Fink, Sciences 285, 251 (1999)). Moreover, the adherence of strains to agar also decreased with ploidy, but could be restored by overexpression of FLO11 (T. Galitski, A. J. Saldanha, C. A. Styles, E. S. Lander, G. R. Fink, Sciences 285, 251 (1999)). It was found herein that the diameter of the mat and the other detailed features of mat architecture decreased as ploidy increased. The alteration in phenotype was dramatic in tetraploid strains, which have four copies of FLO11 but resembled the attenuated, amorphous structure of flo11Δ strains (FIGS. 4A-4D).

The reproducible formation of the yeast floral structure raises important issues concerning the origin of its shape and form. The radial symmetry and the reproducibility of the number of spokes appear to be the hallmarks of a programmed developmental event, but are strongly influenced by the environment. Data presented herein show that the viscosity of the medium, the Flo11p protein on the surface of the yeast cells, and the nutrients in the medium must act in concert for the development of this unique structure.

Although Flo11p is required for both adherence to plastic and mat formation, the molecular basis for this connection is not yet known. One explanation for the role of FLO11 in the adherence of yeast cells to plastic, the multicellular morphological behaviors on 0.3% agar plates, and invasive growth is that Flo11p promotes both cell-cell adhesion and cell-surface adhesion. Previous work has shown that Flo11p is required for cells to adhere to each other in filamentous growth and for cells to adhere to the agar (W. S. Lo and A. M. Dranginis, Mol. Biol. Cell 9, 161 (1998); B. Guo, C. A. Styles, Q. Feng, G. R. Fink, Proc. Nat. Acad. Sci. U.S.A. 97, 12158 (2000)). Flo11p has properties distinct from other yeast cell surface proteins that enable it to initiate biofilm formation. For example, Flo1p, another related cell surface protein, promotes avid cell-cell adhesion but fails to cause adhesion to an inert surface (B. Guo, C. A. Styles, Q. Feng, G. R. Fink, Proc. Nat. Acad. Sci. U.S.A. 97, 12158 (2000)).

Flo11p may play a role similar to that of the M. smegmatis GPLs, which are thought to be required for biofilm formation and sliding function because they increase surface hydrophobicity (J. Recht, A. Martinez, S. Torello, R. Kolter, J. Bacteriol. 182, 4348 (2000); A. Martinez, S. Torello, R. Kolter, J. Bacteriol. 181, 7331 (1999)). Indeed when the hydrophobicity of the FLO11 and flo11Δ strains was measured by their ability to partition between water and octane, it was found that only 12% of the FLO11 cells partitioned to the aqueous phase as compared with 91% of the flo11Δ cells, indicating that FLO11 cells were more hydrophobic. (An aqueous-hydrocarbon biphasic hydrophyobicity assay was adapted from a protocol by (K. C. Hazen and B. W. Hazen, J. Microbiol. Methods 6, 289 (1987)). The cells were grown in liquid SC+2% glucose to an OD₆₀₀ₙₘ of between 0.5-1.5, washed once in water, and resuspended in SC+0.1% glucose to an OD₆₀₀ₙₘ of 0.5. After a stationary incubation of 3 hours at 25° C. the OD₆₀₀ₙₘ of the culture was measured. Then 1.2 mls of the culture were added to a 13×100 mm borosilcate glass tube. The cell suspension was overlaid with 600 μl of octane and the tube was vortexed for 3 min. The phases were allowed to separate and the OD₆₀₀ₙₘ was taken of the aqueous layer. The difference between the OD₆₀₀ₙₘ of the aqueous phase before and after addition of octane was used to determine the hydrophobicity.) The presence of Flo11p might therefore increase the adherence of Saccharomyces to plastic and decrease the adhesion of cells to the more aqueous surface of a 0.3% agar plate. Decreasing the adhesion of the cells to the plate's surface would promote the movement of the cells across the plate. Presumably, the patterns arise from a combination of the frictional forces and the cell-cell interactions. The effect of glucose concentration on the development of these various phenotypes is also likely to be related to the repression of FLO11 transcription by glucose (M. Gagiano, D. van Dyk, F. F. Bauer, M. G. Labrechts, I. S. Pretorius, Mol. Microbiol. 31, 103 (1999)). Reducing the concentration of glucose enhances mat formation and adherence to plastic.

Pathogenic fungi such as Candida albicans and Candida glabrata have orthologs of Flo11p, form mats (Candida albicans formed a mat on 0.3% agar plates with a reproducible morphology that lacked spokes, but had a hub that was distinct from that found in S. cerevisiae mats; the mats formed by C. glabrata lacked hubs and spokes), and C. albicans is known to form biofilms (G. A. O'Toole, H. B. Kaplan, R. Kolter, Annu., Rev. Microbiol. 54, 49 (2000); G. S. Baillie and L. J. Douglas, Methods Enzymol. 310, 644 (1999)). These Flo11p orthologues are thought to be involved in virulence because they confer the ability to adhere to mammalian cells when expressed in Saccharomyces (P. Sundstorm, Curr. Opin. Microbiol. 2, 353 (1999); B. Cormack, N. Ghori, S. Falkow, Science 285, 578 (1999)). Unfortunately, these pathogenic fungi appear to have many redundant copies of the genes encoding these surfaces glycoproteins making it difficult to determine their connection with virulence by mutational analysis (B. Cormack, N. Ghori, S. Falkow, Science 285, 578 (1999); L. L. Hoyer, T. L. Payne, M. Bell, A. M. Myers, S. Scherer, Curr. Genet. 33, 451 (1998)). The discovery that Saccharomyces can undergo the initial steps of biofilm formation indicates that it is a useful model for the genetic dissection of the role of these cell surface proteins in pathogenesis. Compounds that block adhesion will likely prevent the attachment to cells and provide a new avenue to antifungal therapy.

While this invention has been particularly shown and described with reference to preferred embodiments thereof, it will be understood by those skilled in the art that various changes in form and details may be made therein without departing from the scope of the invention encompassed by the appended claims

What is claimed is:

1. A method of producing a fungal biofilm, comprising: culturing a fungus in an appropriate medium that contains a non-glucose repressing carbon source, under conditions appropriate for growth of the fungus, whereby a fungal biofilm is produced.

2. The method of claim 1, wherein the appropriate medium that contains a non-glucose repressing carbon source is a medium in which glucose concentration is limiting.

3. The method of claim 1, wherein the fungus is a pathogenic fungus.

4. The method of claim 3, wherein the pathogenic fungus is a pathogenic yeast.

5. The method of claim 4, wherein the pathogenic yeast is Candida albicans.

6. The method of claim 1, wherein the fungus is a nonpathogenic fungus.

7. The method of claim 6, wherein the nonpathogenic fungus is a nonpathogenic yeast.

8. The method of claim 2, wherein the glucose concentration is about 0.1% or about 0.3%.

9. The method of claim 2, wherein the glucose concentration is from about 0.1% to about 2.0%.

10. The method of claim 9, wherein the glucose concentration is from about 0.1% to about 0.6%.

11. A method of producing a fungal biofilm on a surface, comprising:
(a) applying a fungus in an appropriate medium that contains a non-glucose repressing carbon source to a surface, under conditions appropriate for growth of the fungus, thereby producing a surface having applied thereto a fungus in the appropriate medium; and
(b) maintaining the surface thereby produced under conditions under which fungal cells adhere to the surface and form a biofilm, thereby forming a fungal biofilm on the surface.

12. The method of claim 11, wherein the surface is a solid surface.

13. The method of claim 11, wherein the appropriate medium that contains a non-glucose repressing carbon source is a medium in which glucose concentration is limiting.

14. The method of claim 12, wherein the fungus is a pathogenic fungus.

15. The method of claim 14, wherein the pathogenic fungus is a pathogenic yeast.

16. The method of claim 15, wherein the pathogenic yeast is Candida albicans.

17. The method of claim 11, wherein the fungus is a nonpathogenic fungus.

18. The method of claim 17, wherein the nonpathogenic fungus is a nonpathogenic yeast.

19. The method of claim 13, wherein the glucose concentration is about 0.1% or about 0.3%.

20. The method of claim 13, wherein the glucose concentration is from about 0.1% to about 2.0%.

21. The method of claim 13, wherein the glucose concentration is from about 0.1% to about 0.6%.

22. The method of claim 12, wherein the appropriate medium is synthetic complete medium.

23. The method of claim 11, wherein the surface is a semi-solid surface.

24. The method of claim 23, wherein the fungus is a pathogenic fungus.

25. The method of claim 24, wherein the pathogenic fungus is a pathogenic yeast.

26. The method of claim 25, wherein the pathogenic yeast is Candida albicans.

27. The method of claim 23, wherein the fungus is a nonpathogenic fungus.

28. The method of claim 27, wherein the nonpathogenic fungus is a nonpathogenic yeast.

29. The method of claim 23, wherein the appropriate medium that contains a non-glucose repressing carbon source is a medium in which glucose concentration is limiting and the glucose concentration is 0.1% or about 0.3%.

30. The method of claim 23, wherein the appropriate medium that contains a non-glucose repressing carbon source is a medium in which glucose concentration is limiting and the glucose concentration is from about 0.1% to about 2.0%.

31. The method of claim 23, wherein the appropriate medium that contains a non-glucose repressing carbon source is a medium in which glucose concentration is limiting and the glucose concentration is from about 0.1% to about 0.6%.

32. A fungal biofilm produced by the method of claim 1.

33. A fungal biofilm produced by the method of claim 3.

34. A fungal biofilm produced by the method of claim 4.

35. A fungal biofilm produced by the method of claim 5.

36. A fungal biofilm produced by the method of claim 6.

37. A fungal biofilm produced by the method of claim 7.

38. A fungal biofilm produced by the method of claim 8.

39. A fungal biofilm produced by the method of claim 11.

40. A fungal biofilm produced by the method of claim 12.

41. A fungal biofilm produced by the method of claim 23.

42. A method of identifying a drug that alters formation of a fungal biofilm, comprising:
(a) applying a fungus in an appropriate medium that contains a non-glucose repressing carbon source to a surface, in the presence of a drug to be assessed for its ability to alter formation of a fungal biofilm and under conditions appropriate for growth of the fungus, thereby producing a surface having applied thereto a fungus in the presence of the drug and in the appropriate medium;
(b) maintaining the surface thereby produced under conditions under which fungal cells adhere to the surface and form a biofilm; and
(c) determining the extent to which a fungal biofilm is produced and comparing the extent determined to the extent to which a fungal biofilm is formed under the same conditions but in the absence of the drug, wherein if the extent to which the fungal biofilm is produced is different in the presence of the drug than in the absence of the drug, then the drug is a drug that alters formation of the fungal biofilm.

43. The method of claim 42, wherein the extent to which the fungal biofilm is produced in the presence of the drug is less than the extent to which the fungal biofilm is produced in the absence of the drug and the drug is, therefore, a drug that reduces formation of the fungal biofilm.

44. The method of claim 42, wherein the extent to which the fungal biofilm is produced in the presence of the drug is greater than the extent to which the fungal biofilm is produced in the absence of the drug and the drug is, therefore, a drug that enhances formation of the fungal biofilm.

45. The method of claim 42, wherein the fungus is a pathogenic fungus.

46. The method of claim 42, wherein the fungus is a nonpathogenic fungus.

47. The method of claim 1, wherein the fungus is a filamentous fungus.

48. The method of claim 47, wherein the filamentous fungus is a member of a species selected from the group consisting of: Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species.

49. The method of claim 11, wherein the fungus is a filamentous fungus.

50. The method of claim 49, wherein the filamentous fungus is a member of a species selected from the group consisting of: Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species.

51. The method of claim 12, wherein the fungus is a filamentous fungus.

52. The method of claim 51, wherein the filamentous fungus is a member of a species selected from the group consisting of: Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species.

53. The method of claim 23, wherein the fungus is a filamentous fungus.

54. The method of claim 53, wherein the filamentous fungus is a member of a species selected from the group consisting of: Acremonium species, Aspergillus species, Claviceps species, Colletortichum species, Fusarium species, Monascue species, Neurospora species, Nodulisporium species, Penicillium species, Pestalotiopsis species, Taxomyces species, Tolypocladium species and Trichoderma species.
